(19) United States
(12) Patent Application Publication
(10) Pub. No.: US 2019/0274642 A1
(43) Pub. Date: Sep. 12, 2019

(54) MOVEMENT INDICATOR FOR MOBILE MEDICAL IMAGING APPARATUS

(71) Applicant: Carestream Health, Inc., Rochester, NY (US)

(72) Inventor: Scott T. MacLaughlin, Pittsford, NY (US)

(21) Appl. No.: 15/918,221

(22) Filed: Mar. 12, 2018

(51) Int. Cl. A61B 6/10; A61B 6/00

(52) U.S. Cl. CPC A61B 6/102; A61B 6/4405

(57) ABSTRACT

A mobile x-ray system has a wheeled base for rollably transporting the mobile x-ray system. An x-ray source is attached to a support arm that is attached to the base. A handle on the base is provided to manually maneuver the mobile x-ray system while rollably transporting the system over a surface. A light source attached to the mobile x-ray system projects a visible pattern onto the surface in front of the wheeled base.

### TECHNICAL FIELD

The disclosure relates generally to the field of mobile medical imaging and more particularly to movement indicators for a mobile medical imaging apparatus that is transported from one location to another within a medical care facility.

### BACKGROUND

Medical instrumentation, including devices for various diagnostic and care functions such as medical imaging apparatus, are often designed to be highly portable, allowing an operator or technician to easily transport diagnostic systems from one room or bedside to the next within a medical facility. This feature provides a number of clear advantages for more efficient and effective patient diagnosis and care, but introduces risk factors. Motorized and non-motorized systems, such as mobile x-ray carts, ultrasound systems, and other portable devices can be sizable and difficult to maneuver in tight spaces due to factors such as size, weight, and difficult visibility for single operator movement. Moreover, the traffic environment in a hospital or clinic setting can often be hectic and rapidly changing, with variable amounts of traffic from personnel, moving equipment, patients, and bystanders. Difficulties in movement and maneuverability can be compounded by factors such as narrow hallways, crowded elevators, poor visibility around corners, and other factors. As a result, the risks of injury to operators, patients, or bystanders, or damage to the equipment can be a concern.

One conventional method that has been attempted for such systems uses audible alarm or beeping tones to indicate equipment movement to nearby personnel. However, beeping sounds can be annoying and, even in a crowded hospital atmosphere, may tend to be ignored. Beeping sounds and other audible alarms have limited utility; audible beeps can alert the operator and others nearby to a possible collision, but do not themselves provide any type of feedback that identifies the problem or points to corrective action.

Risk factors such of these have not escaped the attention of regulatory authorities. For example, updated guidelines of ISO 14971, entitled “Medical devices—Application of risk management to medical devices” state that “all risks, regardless of their dimension, need to be reduced as much as possible and need to be balanced, together with all other risks, against the benefit of the device.”

Thus, there is a recognized need for ways to help reduce the risk levels related to the task of safely transporting mobile diagnostic systems, such as diagnostic x-ray imaging apparatus, between locations within a medical care facility.

### SUMMARY

An object of the present disclosure is to advance the art of mobile diagnostic imaging. Embodiments of the present disclosure address the risk factors related to equipment transport described in the background.

A mobile x-ray system has a wheeled base for rollably transporting the mobile x-ray system. An x-ray source is attached to a support arm that is attached to the base. A handle on the base is provided to manually maneuver the mobile x-ray system while rollably transporting the system over a surface. A light source attached to the mobile x-ray system projects a visible pattern onto the surface in front of the wheeled base.

In one embodiment, a mobile x-ray system includes a wheeled base, a support arm attached to the base, and an x-ray source affixed to a distal end of the support arm. A handle attached to the base allows an operator to manually maneuver the mobile x-ray system while rollably transporting it over a floor. A light source attached to the mobile x-ray system projects a visible pattern onto the floor in front of the moving mobile x-ray system.

In one embodiment, motion of a mobile medical imaging apparatus rolling along a surface is detected and a light source is automatically activated in response thereto illuminate an area of the surface in the travel path of the mobile medical imaging apparatus. A detector configured to detect reflected light from the light source is provided to generate a signal in response to detecting the reflected light.

In one embodiment, a mobile x-ray system includes a wheeled base for rolling the mobile x-ray system over a floor, a support arm attached to the base and an x-ray source attached to the support arm. A handle is attached to the base for manually controlling rolling the mobile x-ray system over the floor, and a laser source is attached to the mobile x-ray system to project a visible laser light pattern on the floor in front of the mobile x-ray system while it's being rolled.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a side view of a mobile radiography system.

FIG. 2 is a perspective view that shows a mobile radiography system having a movement indicator apparatus that projects a light pattern indicating the travel path of the apparatus, and shows use of the movement indicator apparatus with a mobile radiography system having an obstructed forward view.

FIG. 3 is a schematic side view that shows components of a movement indicator apparatus provided with a mobile radiography system.

FIG. 4 is a perspective view that shows projection of text having a precautionary or informational message.

FIG. 5 is a perspective view of an embodiment of movement indicator apparatus that uses a single light source mounted on the front of the radiography system.

FIG. 6 is a perspective view that shows use of the sensor for detecting and reporting obstructions in the travel path.

FIG. 7 is a plan view showing a retrofit embodiment.

### DETAILED DESCRIPTION OF THE EMBODIMENTS

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

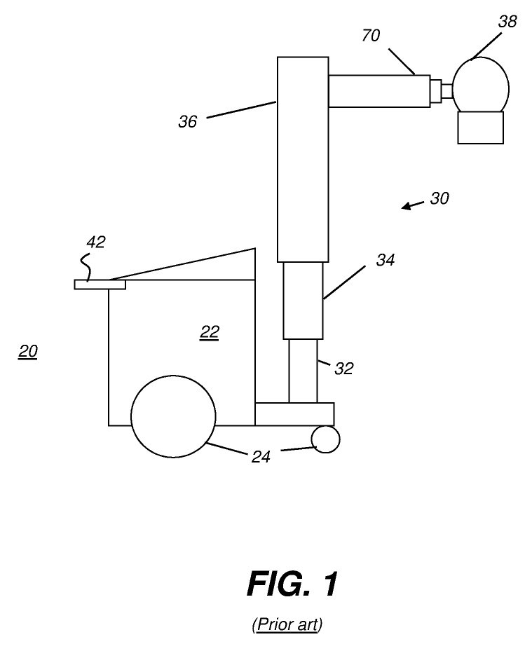

Referring to the side view of FIG. 1, a mobile x-ray or radiography system 20 has a base 22 with wheels 24 for an operator to rollably transport the mobile radiography system over a floor or other travel surface. Base 22 has an extendable and movable support arm 30, 70, attached thereto, which arm 30, 70, may be configured in the form of an extendable column 30 having an adjustable height. An extendable boom 70 is attached to the extendable column 30. Extendable column 30 has telescoping sections 32, 34, and 36 in the embodiment of FIG. 1. An x-ray source 38 is attached to a distal end of the extendable boom 70 of support arm 30, 70, which extendable boom 70 may be extended horizontally from vertical extendable column 30. An operator may control rollable transport of the mobile radiography system 20 using a handle 42 to maneuver the mobile radiography system 20 over a floor or other travel surface. Transport power can be manually provided by the operator or transport can be effected by providing the wheels 24 with a motor drive.

Embodiments of the present disclosure address safety in transporting mobile radiography system 20 over a travel surface from one location to another. Benefits of the apparatus and methods described herein include a capability to clearly demarcate a travel path of the mobile radiography system 20 to signal and/or alert other people as well as to assist the operator to more effectively navigate corners, doorways, elevator entrances, and other tight spaces to help prevent collisions. Advantageously, the movement indicator apparatus disclosed herein can be a built-in feature of the mobile radiography apparatus or it can be retrofit to existing systems, with minimal or no impact on the mechanical profile, maneuverability, or performance of the retrofit system.

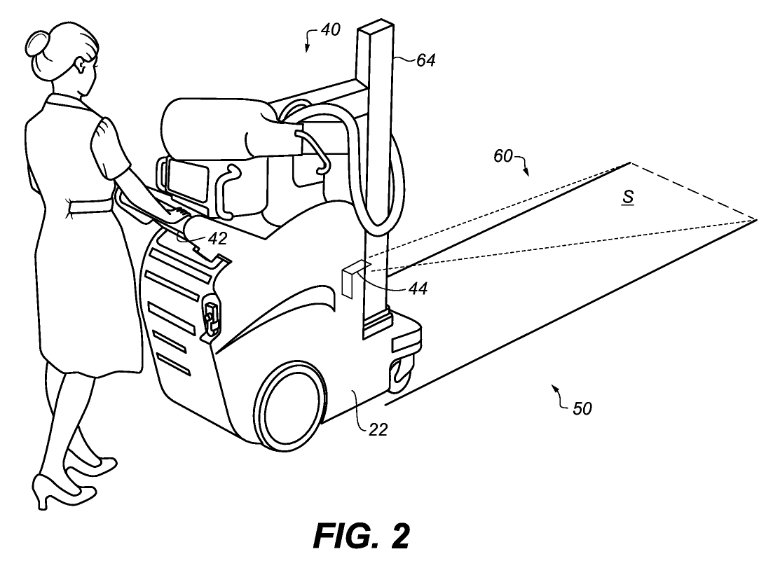

FIG. 2 is a perspective view of a mobile radiography system 40 having a movement indicator apparatus 50 that demarcates the travel path of the mobile radiography system 40 by projecting a light pattern 60 on a travel surface S. One or more light sources 44, such as a line laser source or other low-voltage source that provides a highly visible projection, projects the pattern 60 onto the travel surface S. Light pattern 60 is provided when the mobile radiography system 40 is in motion. Projection light source 44 may be configured to be energized whenever the hands of the operator contact the handle 42 or whenever mobile radiography system 40 is detected to be in motion.

The laser or other light source 44 can project light of any suitable color, including monochromatic, red, green, or blue, for example. A variable colored light source or multiple individually activatable colored lights can be used to indicate various detected conditions. The light source 44 may be controlled to blink or to remain static, as desired. In the embodiment of FIG. 2, the projected pattern 60 from the light source or sources 44 includes a pair of parallel lines that extend forward from, and adjacent to, sides of the base 22. The projected lines can extend in the direction of travel, such as forward or backward as the base 22 is maneuvered by the operator. The pattern 60 can include one or more curved or linear lines projected on the travel surface in front of the base 22 of the mobile radiography system 40, wherein the curved or linear lines are positioned transverse to the pair of parallel lines extend forward from the base 22. For mobile radiography systems 40 having a column 64 that may partially obstruct a forward view, movement indicator embodiments of the present disclosure can be particularly useful.

According to one embodiment of the present invention, light sources of different colors may be energized in response to different detected conditions. For example, a pattern 60 of white or red light may be projected on the travel surface S for indicating standard movement, and a blue light projected when an obstruction or some other condition is detected.

Light source 44 can be provided as a linear laser attached to a side or front surface of the mobile radiography system 40. A linear laser can be provided as a single component, with or without auxiliary lens elements or other light modulators for laser beam shaping. Auxiliary laser beam shaping optics can include various types of lenses such as a cylindrical or Powell lens, holographic lens, apertures, diffraction components, and other devices for conditioning the shape, coverage area, and pattern of the output laser beam. In an alternate embodiment, beam shaping elements can include one or more spatial light modulators that generate and project light patterns onto the travel surface S having graphical and/or text content that can be static or dynamically changing.

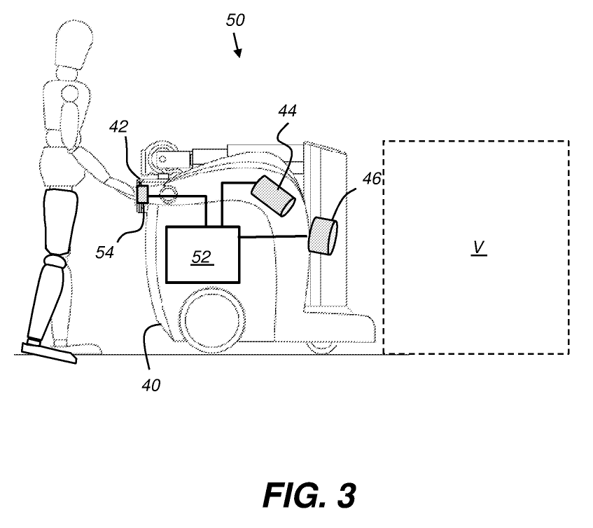

FIG. 3 is a schematic side view of components of a movement indicator apparatus 50 provided with mobile radiography system 40. For aiming and overall orientation of the projected laser pattern content, a volume V, having a width equivalent to a width of the mobile radiography system 40, is selected for marking the travel path of the mobile radiography system 40. Light source 44 is controlled by a control logic processor 52, which can be part of the logic circuitry for operating the mobile radiography system 40 or a separate control logic device. An optional sensor 46 can also be provided for detecting the projected light pattern, as described in more detail subsequently. An input to control logic processor 52 can be a switch 54 that is operatively connected to handle 42 for automatically actuating light source 44 when the handle 42 is touched by an operator or when motion of the mobile radiography system 40 is detected.

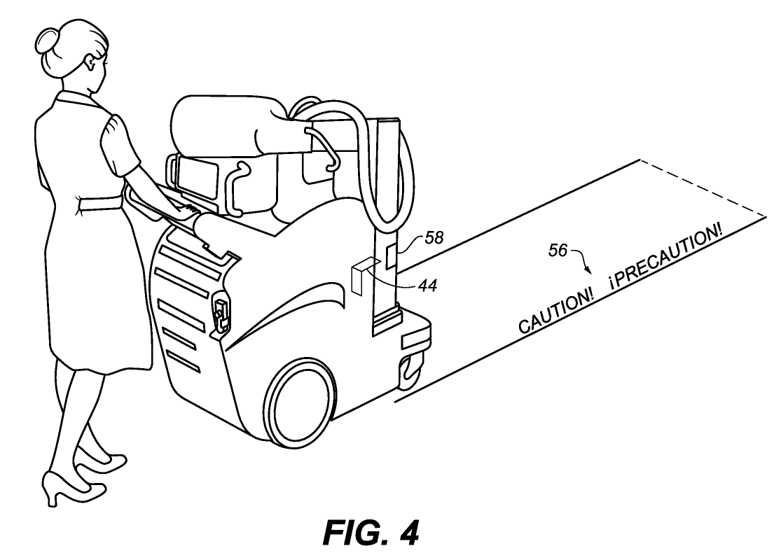

The displayed visible pattern of light can be an outline of volume V projected onto surface S, as shown in FIG. 2. This type of light pattern can be projected using a line laser, without additional lens or modulator components. Alternately, the projected content can be more complex. By way of example, the perspective view of FIG. 4 shows a projected text message 56, such as a precautionary or informational message. Optionally, a logo, hospital or care facility identifier, or equipment or operator identifier could alternately be projected.

With further reference to FIG. 4, a modulator 58 can be provided, particularly where more dynamically changing information is desired to be projected. Modulator 58 can include a spatial light modulator, such as a liquid-crystal device (LCD) or a micro-electromechanical systems (MEMS) device, such as an array of micromirrors or other modulators. One such array can be provided by the Digital Logic Processor (DLP) device from Texas Instruments, Dallas, Tex. A projected scrolling text or animated display generated by modulated light from such a device may attract attention, particularly in crowded, high-traffic corridors.

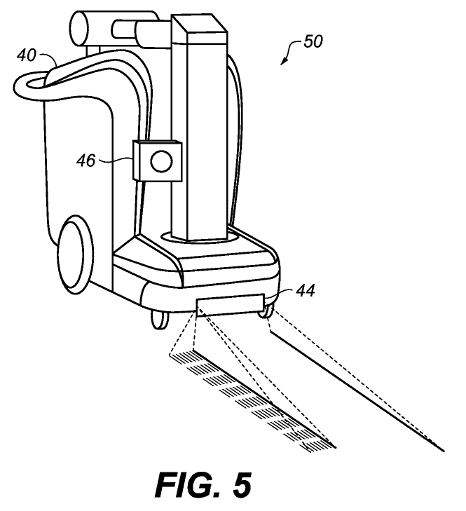

The perspective view of FIG. 5 shows an embodiment of movement indicator apparatus 50 that uses a single light source 44 mounted on a front side of the mobile radiography system 40 to project a light pattern on a travel surface in front of the mobile radiography system 40. Numerous different light patterns can be generated and projected, such as geometric patterns including arcs, arrows, stripes, diagonal lines, etc.

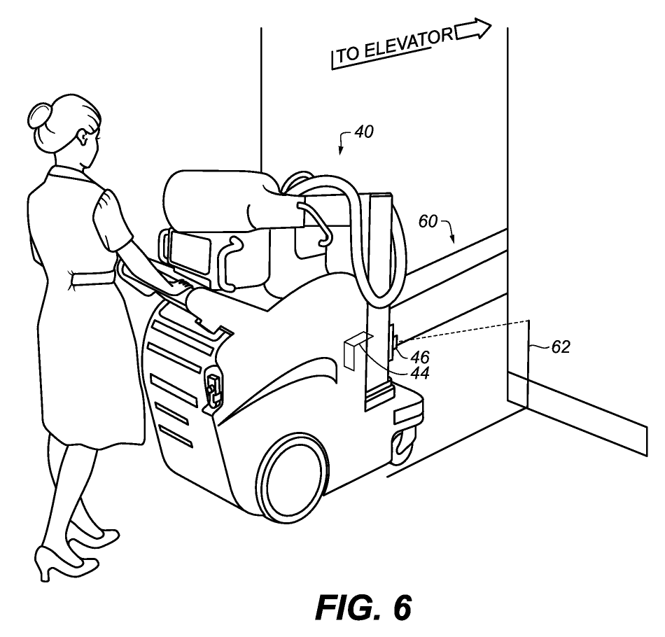

In addition to providing a precautionary display for the operator and others, embodiments of the present disclosure also provide a method for acquiring and assessing feedback information from the displayed content, using the feedback assessment as an indicator of impediments in the travel path. In the perspective view of FIG. 6, sensor 46 functions to detect reflected portions of light pattern 60 to detect any deviation from a normal reflected pattern which indicates a clear, unobstructed travel path. A detected deviated reflected pattern 62 indicates a clearance problem, such as from a nearby obstruction that lies in the intended travel path of the mobile radiography system 40. Control logic processor 52 (FIG. 3) can respond to image analysis from sensor 46 content to generate and transmit a signal that is used to alert the operator to such a condition. A beep, flashing message, flashing projection or other warning indicator can be activated in response to the transmitted signal to warn the operator, or others, to stop or change course in order to avoid a collision or to bypass and clear an obstruction. The projected light can also be configured to change color, such as from green to red, for example, indicating detection of a potential obstruction. A text message indicating detection of an obstruction can be projected. Temporary messages can also alert the operator to slow down or to proceed with caution in a particular location. Intermittent blinking of the light can also be configured to alert the operator and nearby personnel. Image sensing for this purpose can be fairly straightforward, such as configuring sensor 46 to detect some or all portions of reflected light from the projected light pattern 60. Alternative methods for sensing an obstruction can include use of time or frequency encoded projected light patterns to measure time-of-flight, or other light detection algorithms.

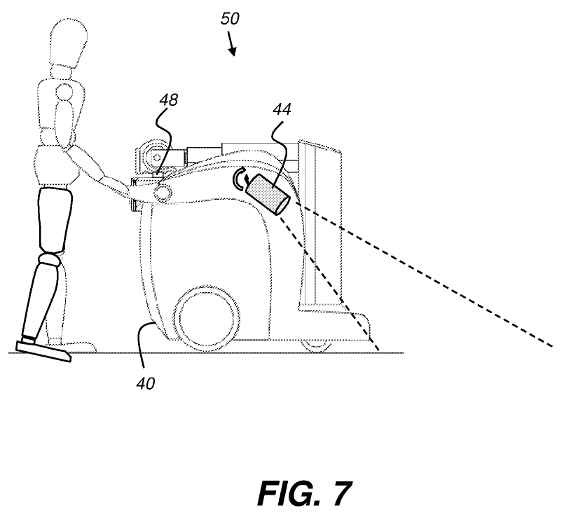

Movement indicator apparatus 50 can also be provided as a retrofit device. The schematic side view of FIG. 7 shows a retrofit arrangement, with light source 44 coupled to a portion of mobile radiography system 40 for providing a precautionary projection device. Magnetic, adhesive, or fastener-based coupling can be used, for example, with the capability provided for variable aiming distance and alignment. An operator switch 48 allows manual activation of movement indicator apparatus 50 in a retrofit device; manual operation can also be provided for actuation of a built-in display system. Power for a retrofit attachment can be provided from connection to a power port on the radiography system or can be provided using standard replaceable batteries, including rechargeable batteries.

As noted previously, the movement indicator apparatus of the present disclosure is ideally suited for support of transport apparatus for a mobile radiography system. In addition, this feature can be provided for other types of mobile equipment, particularly within medical care facilities, where it is desirable to reduce or eliminate risk factors and to help prevent equipment damage or injury. Thus, for example, the movement indicator apparatus of the present disclosure can be used with a mobile ultrasound apparatus, with equipment carts for diagnostic and test systems, with treatment equipment that is designed for portable use, and the like.

According to an alternate embodiment, sensor 46 can be used to detect and report any type of object within the defined volume V (FIG. 3) for the mobile device when in motion. Sensor 46 can be other than a light-sensing device and can use sound or other reflected signal or can be sensitive to heat, such as from nearby personnel or equipment, for example.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A mobile x-ray system comprising:
a base comprising wheels for rollably transporting the mobile x-ray system;
a support arm attached to the base and movable relative to the base;
an x-ray source attached to an end of the support arm opposite the base;
a handle attached to the base, the handle configured to manually maneuver the mobile x-ray system while rollably transporting the mobile x-ray system over a travel surface; and
a light source attached to the mobile x-ray system, wherein the light source projects a visible pattern onto the travel surface.

2. The system of claim 1, wherein the light source comprises a laser source.

3. The system of claim 1, wherein the visible pattern comprises a pair of parallel lines projected adjacent to sides of the base of the mobile x-ray system.

4. The system of claim 3, wherein the visible projection comprises a curved or linear line projected on the travel surface in front of the base of the mobile x-ray system, wherein the curved or linear line is positioned transverse to the pair of parallel lines.

5. The system of claim 1, further comprising means for automatically activating the light source when the mobile x-ray system is transported over the travel surface.

6. The system of claim 1, further comprising means for automatically activating the light source when the handle is touched by an operator.

7. The system of claim 1, wherein the visible pattern comprises an area on the travel surface in front of the base illuminated by the light source.

8. The system of claim 7, wherein the visible pattern comprises a logo.

9. The system of claim 7, wherein the visible pattern comprises human readable text.

10. The system of claim 1, further comprising a sensor to detect light from the visible pattern reflected back to the sensor, the sensor including means to transmit a signal in response to detecting light from the visible pattern.

11. The system of claim 10, further comprising means responsive to the transmitted signal for activating an audible warning, a blinking light, or change of color of the visible pattern.

12. A method comprising:
detecting motion of a mobile medical imaging apparatus along a travel path; and
actuating a light source to project a visible light pattern on the travel path in response to the step of detecting motion.

13. The method of claim 12, further comprising detecting reflected light from the visible light pattern and transmitting a signal in response thereto.

14. A mobile x-ray system comprising:
a wheeled base for rolling the mobile x-ray system over a floor;
a support arm attached to the base;
an x-ray source attached to the support arm;
a handle attached to the base, the handle configured to manually control rolling the mobile x-ray system over the floor; and
a laser source attached to the mobile x-ray system to project a visible laser light pattern on the floor in front of the mobile x-ray system.

15. The system of claim 14, further comprising a light detector to detect the laser light pattern reflected back to the light detector, wherein the laser source is configured to change a color of the laser light pattern in response to a signal from the light detector.

16. The system of claim 14, wherein the visible laser light pattern comprises a pair of parallel lines projected adjacent to sides of the wheeled base of the mobile x-ray system.

17. The system of claim 14, further comprising means for automatically activating the laser source when the mobile x-ray system is in motion over the floor.

18. The system of claim 14, further comprising means for automatically activating the laser source when the handle is touched by an operator.

19. The system of claim 14, wherein the visible laser light pattern comprises human readable text.

20. The system of claim 14, further comprising:
a light detector to detect the laser light pattern reflected back to the light detector; and
a warning indicator that is activated in response to a signal from the light detector.
